(19) United States
(12) Patent Application Publication
(10) Pub. No.: US 2014/0257091 A1
(43) Pub. Date: Sep. 11, 2014

(54) MASTER-SLAVE APPARATUS AND APPROACH

(71) Applicant: The Board of Trustees of the Leland Stanford Junior University, Palo Alto, CA (US)

(72) Inventors: Mark R. Cutkosky, Palo Alto, CA (US); Bruce L. Daniel, Stanford, CA (US); Santhi Elayaperumal, San Francisco, CA (US); Pierre Renaud, Schiltigheim (FR); Yong-Lae Park, Pittsburgh, PA (US)

(21) Appl. No.: 14/204,836

(22) Filed: Mar. 11, 2014

Related U.S. Application Data
(60) Provisional application No. 61/775,876, filed on Mar. 11, 2013.

(51) Int. Cl. B25J 9/00; A61B 19/00; A61B 10/02

(52) U.S. Cl. CPC B25J 9/0033; A61B 10/0266; A61B 19/5244; A61B 2019/5265
USPC 600/424; 74/490.01

(57) ABSTRACT

Aspects of the present disclosure are directed to master-slave apparatuses as well as methods of making and implementing the same. As consistent with one or more embodiments, an apparatus includes a master platform having a manipulation section, and a slave platform mechanically coupled to the master platform and having an interventional-delivery section that secures an interventional tool. The slave platform moves in accordance to three-dimensional movement of the master platform, via supports having a portion thereof fixed relative to the other supports. Each support operates with a respective one of the master and slave platforms for effecting three-dimensional movement of the slave platform, in response to and while tracking (e.g., transmitting) the movement of the master platform, thereby providing control over the interventional tool via the manipulation section of the master platform.

### STATEMENT OF GOVERNMENT SPONSORED SUPPORT

This invention was made with Government support under contract CA159992 awarded by National Institutes of Health. The Government has certain rights in this invention.

### FIELD

Aspects of various embodiments are directed to master-slave devices and related methods.

### BACKGROUND

A variety of approaches have been implemented for effecting the translation of movement for many applications. One such approach involves translating movement for robotic or remote-access type applications, such as for applications involving restricted access and/or where translation of movement can otherwise be beneficial. For instance, interventional applications, such as those involving medical applications, can benefit from remote access.

One type of medical application that involves restricted space is magnetic resonance imaging (MRI), which is an emerging modality for image-guided interventions. Decreasing costs of the technology are making such procedures more feasible. Due to advances in diffusion-weighted (DW) MRI and dynamic contrast-enhanced (DCE) MRI, the selective identification of clinically-significant cancers has also substantially improved. Such advances have led to increased interest in treatments and therapies administered under MRI guidance.

Various robots and positioning apparatuses have been developed for applications such as MRI. However, these devices can be challenging to implement and use for accurate and safe intervention. These and other matters have presented challenges to the translation of movement, and for medical-type interventions, for a variety of applications.

### SUMMARY

Various example embodiments are directed to translational type apparatuses and their implementation.

According to an embodiment, an apparatus includes a master platform having a manipulation section for manipulation by three-dimensional movements, and a slave platform mechanically coupled to the master platform. The slave platform tracks three-dimensional movement of the master platform, and has an interventional-delivery section that secures an interventional tool. The apparatus also includes a plurality of supports, each support having a portion thereof fixed relative to the other supports, and each support being configured with a respective one of the master and slave platforms for effecting three-dimensional movement of the slave platform, in response to and while tracking the movement of the master platform. With this approach, control over the interventional tool is provided via the manipulation section of the master platform.

Another embodiment is directed to a method as may be implemented using the above-noted apparatus. Control over the interventional tool is provided via the manipulation section of the master platform using the plurality of supports, for effecting the three-dimensional movement of the slave platform that is responsive to and tracks movement of the master platform. While providing the control over the interventional tool, tissue with which the interventional tool is engaged is imaged, and the three-dimensional movement of the slave platform is controlled by manipulating the master platform in response to the imaging. With this approach, the slave platform is controlled to mimic movement of the master platform (e.g., allowing remote control of the slave platform via inputs to the master platform, while viewing or otherwise using images obtained via the imaging).

Various embodiments are also directed to monitoring movement of a slave platform as discussed above. These approaches can be implemented, for example, for controlling the apparatus, tool navigation and image-guided movement.

The above discussion/summary is not intended to describe each embodiment or every implementation of the present disclosure. The figures and detailed description that follow also exemplify various embodiments.

### DESCRIPTION OF THE FIGURES

Various example embodiments may be more completely understood in consideration of the following detailed description in connection with the accompanying drawings, in which:

FIG. 1 shows a master-slave apparatus, in accordance with an embodiment of the present disclosure;

FIG. 2 shows an apparatus, as may be implemented with one or more embodiments;

FIG. 3 shows a support structure, as may be implemented in accordance with one or more embodiments of the present disclosure; and

FIG. 4 shows another master-slave apparatus, as may be implemented in accordance with another embodiment of the present disclosure.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Aspects of the present disclosure are believed to be applicable to a variety of different types of apparatuses, systems and methods involving the translation of movement, and the translation of movement for the manipulation of a tool or other device, such as may be implemented for medical intervention. While not necessarily so limited, various aspects may be appreciated through a discussion of examples using this context.

Various example embodiments are directed to a master-slave type device in which a slave component (e.g., a platform) is controlled, via mechanical supports, to mimic three-dimensional movement of a master component. The master and slave are mechanically coupled to a structure via supports, in which a portion of each support is fixed relative to a portion of the other supports. The supports further control movement of the slave to track, or mimic, movement of the master, thereby providing control over the slave. In some implementations, the supports include respective structures connected to each platform, and that are configured relative to one another so that movement of the slave is restricted to movement that mimic movement of the master. In one such implementation, master/slave platforms are connected maintaining a distance relative to one another, and the respective supports connected to each platform operate to maintain orientation of the slave platform relative to the master platform, (e.g., with 1:1 or at another ratio of movement). In certain embodiments, aspects of the supports are asymmetric, as may be implanted radially, within a single master or slave structure, or relative to the other of the master and slave structures.

As discussed herein, one or more embodiments directed to the tracking of movement of the master platform, by the slave platform, may relate to one or more aspects of sensing and/or reacting to a change in position of the master platform and structures connected thereto. As such, various embodiments are directed to tracking movement in this regard, which may generally mimic movement at one or more of a variety of spatial and force ratios. In this context, the master platform transmits motion to the slave platform, and the slave platform effectively senses and responds to the configuration of the master platform. Similarly, one or more embodiments relating to the translation of movement or force may involve a variety of slave three-dimensional movements as may generally mimic those movements of the master, such as rotational movement, linear movement and others.

Another embodiment is directed to an apparatus including a master platform having a manipulation section and a slave platform mechanically coupled to the master platform, such as by supports extending between the platforms. Each platform is connected to two or more support structures specific to each platform, with the support structures being fixed relative to the other support structures. The support structures limit or otherwise control movement of the respective platforms relative to one another such that the slave platform tracks, or mimics, three-dimensional movement of the master platform in response to manipulation of the master platform. Such an approach facilitates remote mechanical control of the slave platform, such as for controlling the movement and positioning of an interventional tool or other object connected to the slave platform.

In a more particular embodiment, the supports include two or more prismatic joints, and respective sliders that are coupled to one of the prismatic joints and fixed relative to the other sliders. Each slider operates to slide along the prismatic joints, and is connected by struts to one of the master or slave platform. The struts, sliders and prismatic joints operate to control the slave platform to track/mimic movement of the master platform. For instance, as the master platform is moved, respective struts connected to sliders move relative to one another, with each slider causing relative movement of sliders connected to the slave platform via coupling by the prismatic joints.

In certain implementations, the struts, sliders and prismatic joints control the slave platform to mimic the three-dimensional movement of the master platform at a ratio of movement defined by symmetry characteristics of the struts (i.e., including both symmetry and asymmetry), sliders and prismatic joints, relative to each of the master platform and the slave platform. For instance, asymmetric struts can be used to limit movement of the master platform, with similarly asymmetric struts being used to limit movement of the slave platform such that movement of the respective platforms track one another. In some implementations, the asymmetric struts scale forces applied to the master relative to the slave, such that the slave applies more or less force than applied via the master.

The supports may include one or more of a variety of types of mechanical structures, which can be tailored to suit particular applications and available materials. In some embodiments, the supports include prismatic joints such as discussed above, and movement of the slave platform is controlled relative to the master platform in a dimension set via the joints. In some embodiments, one or both platforms are attached to first and second supports that are fixed relative to one another and that are radially asymmetric relative to one another and the platform. In certain embodiments, one or both platforms are connected via first and second supports that are asymmetric relative to one another. In still other embodiments, supports connected to the master platform are asymmetric relative to supports connected to the slave platform.

Another more particular embodiment is directed to a master-slave apparatus as above, with a master gimbal connected to the master platform and a slave gimbal connected to the slave platform. The gimbals are connected via connectors extending between the master gimbal and the slave gimbal, and the connectors are used to control movement of the slave gimbal relative to the slave platform, to track movement of the master gimbal relative to the master platform.

The master-slave apparatus as discussed herein may be implemented in a variety of manners, for many applications such as those discussed in the background above and in the underlying provisional patent application to which benefit is claimed, as well as in the references cited therein, all of which are fully incorporated herein by reference. For instance, the slave may be controlled via a master for remote intervention with a variety of objects, or patients. In some embodiments, a master-slave apparatus as discussed above translates force to provide a touch, or feel-type feedback to a user or machine manipulating the master. In one such particular embodiment, the slave platform and supports operate to present a force to the master platform that is responsive to interaction between a tool secured to the slave platform and a subject. Such an approach may be implemented for intervention with tissue, such as for surgery or other type operations, which may be facilitated for remote access by the master/slave apparatus. Such a translated force may be indicative of a reactionary force applied to a tool in response to movement of the tool controlled via movement of the master platform, which can provide a sense of pressure applied by the tool to tissue.

Various embodiments are directed to methods involving use of a master-slave type apparatus as discussed herein along with imaging, in which an interventional tool connected to a slave platform is controlled via manipulation of a master platform, using respective supports to limit movement of the slave platform relative to the master platform. Tissue within a specimen is imaged, such as via MRI, CT (x-ray computed tomography), PET (positron emission tomography), US (ultrasound) or other imaging approaches. The interventional tool is engaged with the tissue by manipulating the master platform, therein controlling three-dimensional movement of the slave platform and the tool.

Accordingly, various aspects of the disclosure are directed to providing a low-friction, lightweight passive device for tracking (e.g., 1:1 or other ratio mapping) of rotations and translations through a set of parallel mechanical linkages. In some embodiments, such an apparatus is based on a double Delta parallel mechanism having two ends (master and slave) connected by a set of three prismatic linkages, providing two P-U-U (prismatic-universal-universal) parallel kinematic chains. The device is made to a length (via the prismatic linkages) that suits particular applications. In some implementations, connected gimbals are used in each platform to decouple platform rotations from translations in the X, Y and Z directions.

Various embodiments are directed to implementation of a master-slave type device as discussed herein, without a fixed base, to facilitate an infinite workspace in an insertion depth of field (DOF) (Z-direction). Rotational degrees of freedom are decoupled from translational degrees of freedom to facilitate kinematic modeling and precise control of the position and orientation of the master and slave platforms. Such a device may be implemented without power, and thus is quiet and can be implemented in application in which electromagnetic, wireless or radio frequency (RF) interference may be an issue.

One such embodiment involves implementation within an MRI-type device, with the master being used at a portion near an entry or external to the MRI-type device and the slave being used within the device. In certain embodiments, the device is implemented with asymmetric components that facilitate access in an MRI machine, such as in manners shown in the Figures. A similar approach can be used with a variety of types of intervention devices, in addition to MRI devices, such as those that can be used to facilitate guided interventions or minimally invasive procedures including biopsy, brachytherapy, and cryosurgery, and can be used in adverse environments (e.g., where remote manipulation is useful due to safety concerns, such as under water or to avoid high radiation doses).

In a particular MRI application, a master-slave mechanism as discussed herein is used to allow a physician standing just outside the bore of an MRI scanner to intuitively manipulate an interventional tool, such as a biopsy needle, inside the bore. This can be used, for example, for transperineal MRI-guided prostate biopsy, brachytherapy and cryotherapy. The manipulator is used in conjunction with an interactive imaging system, which relays real-time information about instrumented tools, such as their 3D shape and interaction forces. The apparatus provides physician access to the patient, even in close-bore MRI machines, and could be integrated with advanced systems. In some implementations, a tool (e.g., needle) position is monitored in real-time, such as by using RF (radio frequency) or EM (electro-magnetic) tracking coils, fiducial markers, acoustic or optically based sensors with an MRI application that are embedded in a platform or other aspect of the tool.

In addition, various embodiments are directed to such a master-slave mechanism with one or more additional and/or fewer aspects. In some embodiments, external apparatuses are implemented to permit motion in six degrees of freedom, including bearings, Lazy-Susan devices and linear stages. In certain embodiments, such a master-slave mechanism is replicated and contained within a larger master-slave mechanism of the same design, facilitating two-handed operation and articulation of different points of an end-effector or tool. In other embodiments, certain approaches using Delta mechanisms are implemented with Stewart mechanisms to track movement, such as to couple translations and rotations. In some embodiments, counter-balancing and braking are implemented with a master-slave device as discussed herein, to facilitate desirable platform positioning between manipulations. Other non-symmetric shapes relative to those shown here are also implemented in various embodiments, for various symmetry aspects (e.g., of a cross-section of mechanisms herein). Further, the various components discussed herein can be made of one or more types of materials in different embodiments, such as nonconductive, non-metal, or non-ferrous materials, which can be tailored to suit the application and use environment. Linkage lengths and platform sizes are varied to suit respective embodiments, such as to scale input-to-output displacements and forces.

In some embodiments, master and slave platforms or related support structures are implemented with measures to facilitate motion control. In some embodiments, counter-balancing components such as rubber-bands, carbon leaf springs, and torsion springs are used. For instance, a torsion spring placed on a slider at a unique angle relative to the manipulator and gravity can counteract the weight of a platform and balance the platform. Such a moment arm can be calculated from Jacobian-based points (e.g., as discussed in the provisional patent application referenced herein). In more particular embodiments, braking is used to restrict or otherwise control movement of platforms as discussed herein. For instance, fixing or braking slider positions restricts the platforms from moving, thus braking an end-effector tool connected to a platform. Some aspects involve pneumatic braking, such as by using medical air lines (e.g., available in an MRI scanner).

Various embodiments are also implemented in accordance with, using or otherwise involving one or more aspects of the underlying provisional application to which priority is claimed, including embodiments, further teachings and/or examples as described in Appendices A, B and C that form part of the provisional patent application. Moreover, one or more embodiments may be implemented in accordance with or otherwise using approaches described in one or more of the various references cited in the provisional patent application.

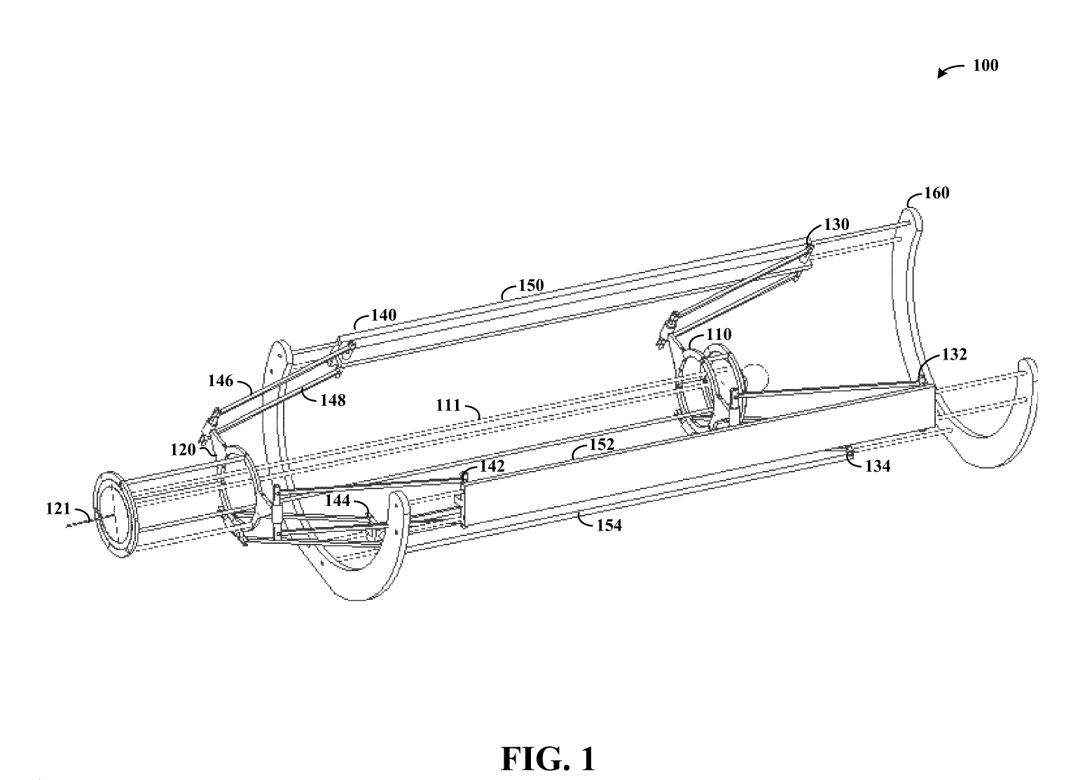

Turning now to the figures, FIG. 1 shows a master-slave apparatus 100, in accordance with another embodiment of the present disclosure. The master-slave apparatus 100 includes a master platform 110 and a slave platform 120, with an end-effector type tool 121 connected to the slave platform. Master platform 110 is connected to sliders 130, 132 and 134, and slave platform 120 is connected to sliders 140, 142 and 144. Struts connect each slider with its respective master or slave platform, with struts 146 and 148 as connected to the slave platform being labeled by way of example. The sliders are respectively coupled to prismatic joints 150, 152 and 154. The prismatic joints are affixed to a frame 160, the shape of which may be tailored to suit applications, as may be the symmetry (or asymmetry) of respective aspects of the platforms and struts.

The master and slave platforms 110 and 120 are optionally coupled to one another via struts, with strut 111 shown by way of example. Movement of the master platform 110 is translated via the struts to the slave platform 120, with three-dimensional movement of the slave platform being tied to track three-dimensional movement of the master platform by way of the struts and sliders, as well as their respective arrangements. Further, the platforms may be implemented with gimbals that provide additional degrees of freedom.

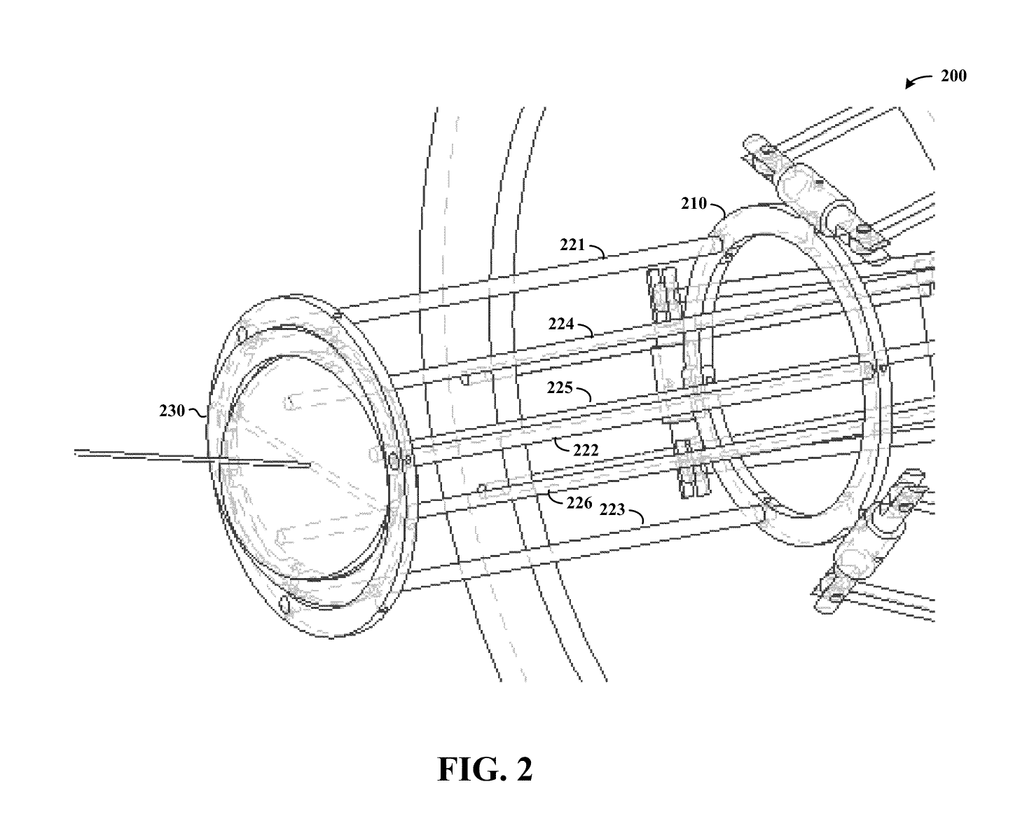

FIG. 2 shows an apparatus 200, as may be implemented with one or more embodiments (e.g., as with the master-slave apparatus 100 in FIG. 1). The apparatus 200 includes a support structure 210 with multiple struts including frame support struts 221-223 that are coupled to (or form part of) the support structure as discussed above, as well as struts 224-226 that couple a gimbal-type structure 230 to a manipulating structure on a master platform (e.g., similar to the gimbal-type structure). The struts operate to mimic movement of the support structure 210 relative to movement of the master platform, such as via two axes as shown.

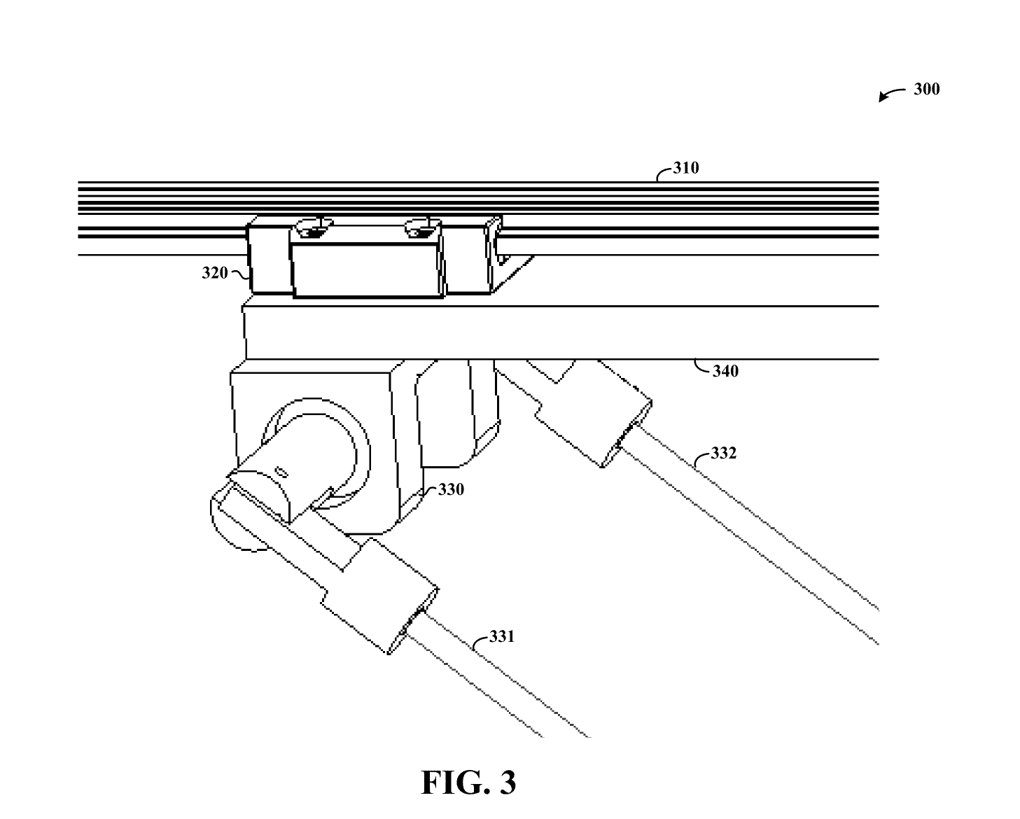

FIG. 3 shows a support structure 300, as may be implemented in accordance with one or more embodiments of the present disclosure. The support structure 300 includes a prismatic sliding joint including a track 310 and slider 320 that provides slidable motion relative to the track. A joint 330 is shown coupled to struts 331 and 332, which can be coupled to a master or slave platform. The slider 320 is connected by a connector 340 to another slider that is connected to the other of the master or slave platforms. The support structure 300 may, for example, be implemented with a master-slave apparatus such as shown in FIG. 1, with multiple such structures used and connected to one another to facilitate the transmission and tracking of movement in the slave platform, relative to the master platform.

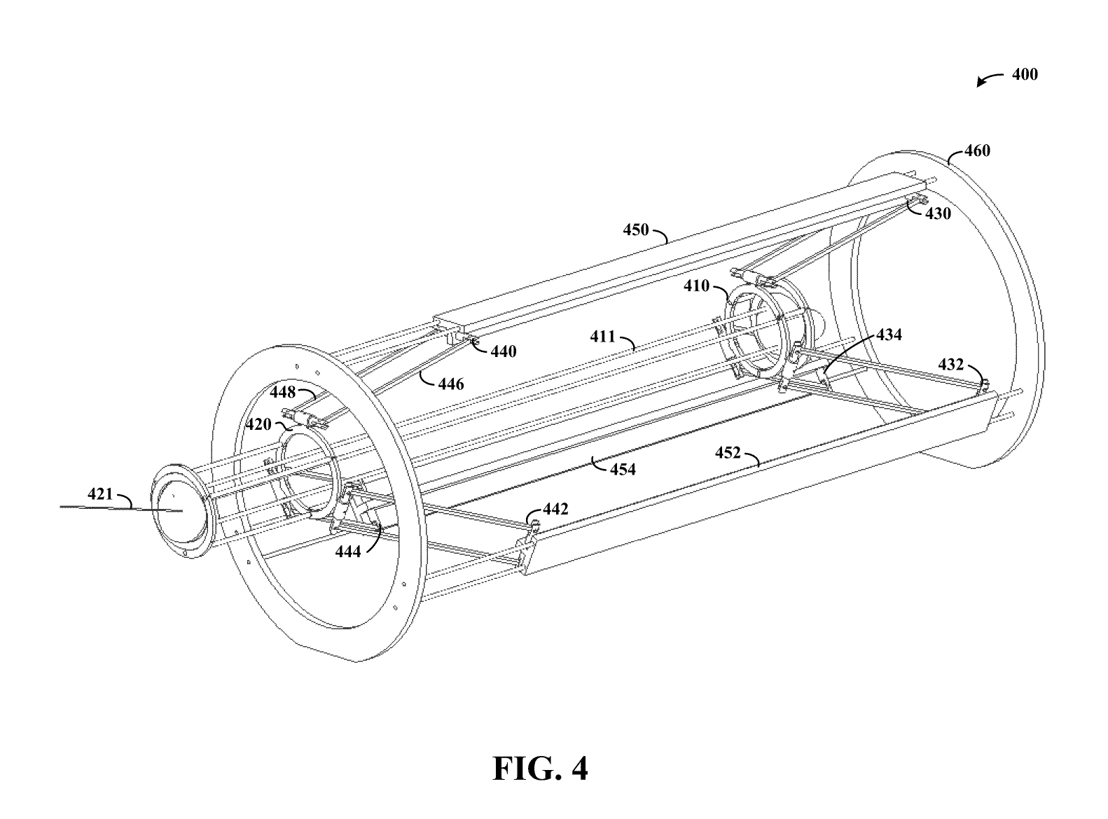

FIG. 4 shows another master-slave apparatus 400, as may be implemented in accordance with another embodiment of the present disclosure. The master-slave apparatus 400 is similar to the master-slave apparatus 100 shown in FIG. 1, and includes a master platform 410 and a slave platform 420 mounted to a frame 460, with an end-effector type tool 421 connected to the slave platform. Master platform 410 is connected to sliders 430, 432 and 434, and slave platform 420 is connected to sliders 440, 442 and 444, with the sliders being mounted on a common support. Struts connect each slider with its respective master or slave platform, with struts 446 and 448 connected to the slave platform 420 being labeled by way of example. The supports on which the sliders are mounted are respectively coupled to prismatic joints 450, 452 and 454. Gimbals on the master and slave platforms 410 and 420 are also coupled to one another via struts, with strut 411 shown by way of example. Movement of the master platform 410 is translated via the struts and prismatic joints to the slave platform 420, with three-dimensional movement of the slave platform being tied to track three-dimensional movement of the master platform by way of the struts and sliders, as well as their respective arrangements.

Various blocks, modules or other circuits may be implemented to carry out one or more of the operations and activities described herein and/or shown in the figures. In these contexts, a “block” (also sometimes “logic circuitry” or “module”) is a circuit that carries out one or more of these or related operations/activities (e.g., controlling movement of a master gimbal and/or master platform, tracking, or imaging). For example, in certain of the above-discussed embodiments, one or more modules are discrete logic circuits or programmable logic circuits configured and arranged for implementing these operations/activities. In certain embodiments, such a programmable circuit is one or more computer circuits programmed to execute a set (or sets) of instructions (and/or configuration data). The instructions (and/or configuration data) can be in the form of firmware or software stored in and accessible from a memory (circuit). As an example, first and second modules include a combination of a CPU hardware-based circuit and a set of instructions in the form of firmware, where the first module includes a first CPU hardware circuit with one set of instructions and the second module includes a second CPU hardware circuit with another set of instructions.

Certain embodiments are directed to a computer program product (e.g., nonvolatile memory device), which includes a machine or computer-readable medium having stored thereon instructions which may be executed by a computer (or other electronic device) to perform these operations/activities.

Based upon the above discussion and illustrations, those skilled in the art will readily recognize that various modifications and changes may be made to the various embodiments without strictly following the exemplary embodiments and applications illustrated and described herein. For example, additional or fewer degrees of freedom may be implemented with respect to various embodiments, and differently shaped connectors and support structures may also be implemented. In addition, the various embodiments described herein may be combined in certain embodiments, and various aspects of individual embodiments may be implemented as separate embodiments. Such modifications do not depart from the true spirit and scope of various aspects of the invention, including aspects set forth in the claims.

What is claimed is:

1. An apparatus comprising:
a master platform having a manipulation section configured and arranged to be manipulated by three-dimensional movements;
a slave platform mechanically coupled to the master platform and configured and arranged to track three-dimensional movement of the master platform, and having an interventional-delivery section configured and arranged to secure an interventional tool; and
a plurality of supports, each support connected to the master platform having a portion thereof fixed relative to another one of the supports connected to the slave platform, and each support being configured and arranged with a respective one of the master and slave platforms for effecting three-dimensional movement of the slave platform, in response to and while tracking the movement of the master platform, thereby providing control over the interventional tool via the manipulation section of the master platform.

2. The apparatus of claim 1, wherein the plurality of supports includes:
a plurality of prismatic joints,
a plurality of sliders, each slider coupled to one of the prismatic joints and being fixed relative to another slider coupled to the same prismatic joint, each slider being configured and arranged to slide along the prismatic joints and the prismatic joints being configured and arranged to move relative to the other prismatic joints, and
for each platform, first and second struts respectively connected between the platform and different ones of the plurality of sliders, the struts being configured and arranged with the sliders and prismatic joints to control the slave platform to track the movement of the master platform.

3. The apparatus of claim 2, wherein the struts, sliders and prismatic joints are configured and arranged to control the slave platform to mimic the three-dimensional movement of the master platform at a ratio of movement defined by symmetry characteristics of the struts, sliders and prismatic joints, relative to each of the master platform and the slave platform.

4. The apparatus of claim 1, wherein the plurality of supports includes a plurality of prismatic joints, the supports being coupled to and configured and arranged with the prismatic joints to control the slave platform to move relative to the master platform.

5. The apparatus of claim 1, wherein the plurality of supports includes, for at least one of the platforms, first and second supports that are fixed relative to one another and that are radially asymmetric relative to one another and the platform.

6. The apparatus of claim 1, wherein the plurality of supports includes, for at least one of the platforms, first and second supports that are asymmetric relative to one another.

7. The apparatus of claim 1, wherein the plurality of supports includes a first support connected to the master platform and a second support connected to the slave platform, the first and second supports being asymmetric relative to one another.

8. The apparatus of claim 1, further including
a master gimbal connected to the master platform;
a slave gimbal connected to the slave platform; and
a plurality of connectors connected between the master gimbal and the slave gimbal and configured and arranged to control movement of the slave gimbal relative to the slave platform to track movement of the master gimbal relative to the master platform.

9. The apparatus of claim 1, wherein the slave platform is configured and arranged with the plurality of supports to present a force to the master platform, in response to interaction between a tool secured to the interventional-delivery section and a subject, the force being indicative of a reactionary force applied to the tool in response to movement of the tool controlled via movement of the master platform.

10. A method comprising:
manipulating a slave platform mechanically coupled to a master platform having a manipulation section configured and arranged to be manipulated by three-dimensional movements, the slave platform being configured and arranged to track three-dimensional movement of the master platform, and having an interventional-delivery section configured and arranged to secure an interventional tool, by
providing control over the interventional tool via the manipulation section of the master platform using a plurality of supports, each support connected to the master platform having a portion thereof fixed relative to another one of the supports connected to the slave platform, each support being configured and arranged with a respective one of the master and slave platforms for effecting three-dimensional movement of the slave platform, to effect three-dimensional movement of the slave platform that is responsive to and tracks movement of the master platform; and
while providing the control over the interventional tool, imaging tissue with which the interventional tool is engaged, and controlling three-dimensional movement of the slave platform by manipulating the master platform in response to the imaging, thereby implementing the slave platform to mimic movement of the master platform.
